(19) United States
(12) Patent Application Publication
(10) Pub. No.: US 2020/0375568 A1
(43) Pub. Date: Dec. 3, 2020

(54) AUXILIARY ELECTROCARDIOGRAM (ECG) ASSEMBLIES AND CLINICAL DATA ACQUISITION SYSTEMS INCLUDING AUXILIARY ECG ASSEMBLIES

(71) Applicant: EchoNous, Inc., Redmond, WA (US)

(72) Inventors: Nikolaos Pagoulatos, Kirkland, WA (US); David Nelson, Redmond, WA (US); Greg Nieminen, Bothell, WA (US); Shelby Brunke, Sammamish, WA (US); Michael Blaivas, Alpharetta, GA (US); Terry Doherty, Snohomish, WA (US); Ron Broad, Arlington, WA (US); Jeff Petermann, Snohomish, WA (US)

(21) Appl. No.: 16/888,366

(22) Filed: May 29, 2020

Related U.S. Application Data
(60) Provisional application No. 62/854,931, filed on May 30, 2019.

(51) Int. Cl. A61B 8/02; A61B 8/00

(52) U.S. Cl. CPC A61B 8/02; A61B 8/4254; A61B 8/4455; A61B 2562/227; A61B 8/4236; A61B 2560/0431; A61B 2562/02; A61B 8/4472

(57) ABSTRACT

Devices and systems are provided that include an auxiliary electrocardiogram (ECG) assembly that can be utilized to acquire ECG data of a patient and transmit the ECG data to a handheld probe, such as an ultrasound probe. The handheld probe may include a housing and an ultrasound sensor that is at least partially surrounded by the housing. The ultrasound sensor may be located at or near a sensor face of the handheld probe. An auxiliary ECG connector is included that is at least partially exposed by the housing of the handheld probe. The auxiliary ECG connector may be utilized to electrically couple one or more auxiliary ECG leads to the handheld probe.

### BACKGROUND

### Technical Field

The present application pertains to medical monitoring, and more particularly to ultrasound systems including auxiliary electrocardiogram (ECG) leads.

### Description of the Related Art

Ultrasound imaging is a useful imaging modality in a number of environments. For example, in the field of healthcare, internal structures of a patient's body may be imaged before, during or after a therapeutic intervention. A healthcare professional may hold a portable ultrasound probe, or transducer, in proximity to the patient and move the transducer as appropriate to visualize one or more target structures in a region of interest in the patient. A transducer may be placed on the surface of the body or, in some procedures, a transducer is inserted inside the patient's body. The healthcare professional coordinates the movement of the transducer so as to obtain a desired representation on a screen, such as a two-dimensional cross-section of a three-dimensional volume.

Ultrasound imaging is typically performed in a clinical setting, by trained ultrasound experts, utilizing ultrasound systems that are specifically designed to acquire ultrasound data. Similarly, electrocardiography (ECG) is typically performed in a clinical setting by trained experts and utilizing equipment that is specifically designed for acquiring electrocardiography data.

Acquisition of these different types of clinical data, i.e., ultrasound data and ECG data, is thus conventionally performed utilizing separate pieces of equipment, and often in separate patient visits or separate environments.

For many years, ultrasound imaging was effectively confined to large equipment operating in a hospital environment. Recent technological advances, however, have produced smaller ultrasound systems that increasingly are deployed in frontline point-of-care environments, e.g., doctor's offices.

### BRIEF SUMMARY

The present application, in part, addresses a desire for smaller clinical data acquisition systems, such as ultrasound and electrocardiogram (ECG) systems, having greater portability, lower cost, and ease of use, while at the same time providing high quality measurements. Further, the present application, in part, addresses a desire for clinical data acquisition systems, such as ultrasound systems, having a probe that may be electrically or communicatively coupled to an auxiliary ECG assembly having ECG electrodes and which is capable of sensing ECG signals of a patient while simultaneously acquiring ultrasound images.

In at least one embodiment, a handheld probe includes a housing and an ultrasound sensor that is at least partially surrounded by the housing. An auxiliary ECG connector is included as part of the handheld probe and is at least partially exposed by the housing. The auxiliary ECG connector is configured to electrically couple one or more ECG leads to the handheld probe.

In at least one embodiment, a clinical data acquisition device is provided that includes a handheld probe and an auxiliary ECG assembly. The handheld probe includes at least one sensor configured to acquire physiological data of a patient. The auxiliary ECG assembly includes a plurality of ECG leads configured to acquire ECG data of the patient. The auxiliary ECG assembly is communicatively coupleable to the handheld probe.

In at least one embodiment, a clinical data acquisition system is provided that includes a handheld probe, an auxiliary ECG assembly, and a mobile clinical viewing device. The handheld probe includes at least one sensor configured to acquire physiological data of a patient. The auxiliary assembly includes a plurality of ECG leads configured to acquire ECG data of the patient, and the auxiliary ECG assembly is communicatively coupleable to the handheld probe. The mobile clinical viewing device is communicatively coupled to the ultrasound probe, and the mobile clinical viewing device includes a display configured to display the acquired physiological data of the patient and the acquired ECG data of the patient.

### BRIEF DESCRIPTION OF THE SEVERAL VIEWS OF THE DRAWINGS

FIG. 1 is a perspective view illustrating a clinical data acquisition system that includes a mobile clinical viewing device and a clinical data acquisition probe, in accordance with one or more embodiments of the present disclosure.

FIG. 2 is a perspective view illustrating the clinical data acquisition probe of the clinical data acquisition system shown in FIG. 1, in accordance with one or more embodiments.

FIG. 3 is a perspective view illustrating an auxiliary ECG assembly connected to the clinical data acquisition probe shown in FIG. 2, in accordance with one or more embodiments.

FIG. 4 is a diagram illustrating another auxiliary ECG assembly connected to a clinical data acquisition probe, in accordance with one or more embodiments.

FIG. 5 is a diagram illustrating another auxiliary ECG assembly which may be connected to a clinical data acquisition probe, in accordance with one or more embodiments.

FIG. 6 is a diagram illustrating another auxiliary ECG assembly which may be wirelessly connected to a clinical data acquisition probe, in accordance with one or more embodiments.

FIG. 7 is a diagram illustrating a clinical data acquisition system including a wireless auxiliary ECG assembly, in accordance with one or more embodiments.

FIG. 8A is a diagram illustrating magnetic connectors for coupling auxiliary ECG assemblies to a clinical data acquisition probe, in accordance with one or more embodiments.

FIG. 8B is a diagram illustrating snap-on type connectors for coupling auxiliary ECG assemblies to a clinical data acquisition probe, in accordance with one or more embodiments.

FIG. 9 is a diagram illustrating a snap-on type connector for electrically coupling auxiliary ECG leads to ECG electrodes located at a sensor face of a clinical data acquisition probe, in accordance with one or more embodiments.

FIG. 10 is a diagram illustrating a clinical data acquisition system including an auxiliary ECG assembly coupled between a mobile clinical viewing device and a clinical data acquisition probe, in accordance with one or more embodiments.

FIG. 11 is a diagram illustrating a clinical data acquisition probe including an auxiliary ECG electrode connector, in accordance with one or more embodiments.

FIG. 12 is a diagram illustrating a mobile clinical viewing device including an auxiliary ECG electrode connector, in accordance with one or more embodiments.

### DETAILED DESCRIPTION

Three primary techniques used extensively in medicine for physiological assessment, e.g., of the cardiothoracic cavity, include sonography, auscultation, and electrocardiography. Each technique provides different kinds of information usable to assess the anatomy and physiology of the organs present in a region of interest, e.g., the cardiothoracic cavity.

Medical ultrasound imaging (sonography) has been one of the most effective methods for examining both the heart and the lungs. Ultrasound imaging provides anatomical information of the heart as well as qualitative and quantitative information on blood flow through valves and main arteries such as the aorta and pulmonary artery. One significant advantage of ultrasound imaging is that, with its high frame rate, it can provide dynamic anatomical and blood flow information which is vital for assessing the condition of the heart which is always in motion. Combined with providing blood flow information, ultrasound imaging provides one of the best available tools for assessing the structure and function of heart chambers, valves, and arteries/veins. Similarly, ultrasound imaging can assess fluid status in the body and is the best tool in assessing pericardial effusion (fluid around the heart).

In the case of lungs, ultrasound imaging provides information on the anatomical structure of the lungs with the ability to show specific imaging patterns associated with various lung diseases and with an ability to assess fluid status around the lung and within individual compartments of the lung including the assessment of pericardial effusion.

Auscultation allows for assessing the physiological condition and function of organs such as the heart and lungs by capturing audible sounds that are produced by or otherwise associated with these organs. The condition and function of these organs, or other organs as the case may be, can be evaluated based on clinical information indicating how different sounds are associated with various physiological phenomena and how the sounds change for each pathological condition.

Electrocardiography (EKG or ECG) is focused on the heart by capturing the electrical activity of the heart as it is related to the various phases of the cardiac cycle. The condition and function of the heart may be evaluated based on clinical knowledge indicating how the electrical activity of the heart changes based on various pathological conditions.

The present disclosure provides systems, devices, and methods in which auxiliary ECG assemblies and electrodes are operable to communicate with a handheld probe, and the handheld probe in turn is is operable to acquire ultrasound and ECG signals using the auxiliary ECG electrodes. In some embodiments, the handheld probe is further operable to acquire auscultation signals.

In some embodiments, some or all of these three types of signals (i.e., auscultation, ECG, and ultrasound signals) are synchronously acquired and displayed via one or more audiovisual outputs. Providing a combination of two or more of auscultation, ECG, and ultrasound data significantly enhances the ability of doctors and others to accurately and efficiently assess the physiological condition of a patient, especially of the patient's heart and lungs.

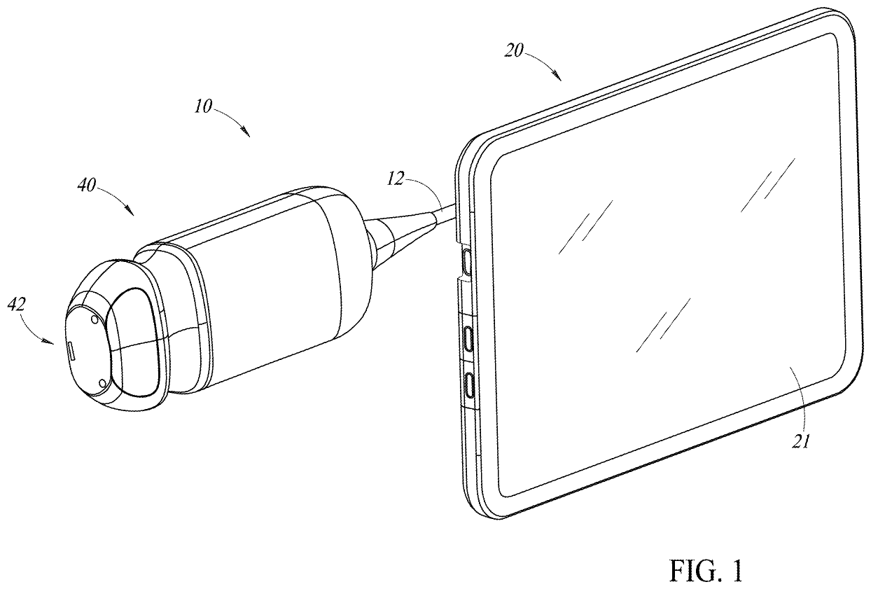

FIG. 1 is a schematic illustration of a clinical data acquisition system 10, in accordance with one or more embodiments of the present disclosure. The clinical data acquisition system 10 includes a mobile clinical viewing device 20 (which may be referred to herein as tablet 20) and a clinical data acquisition probe 40 (which may be an ultrasound probe and may be referred to herein as ultrasound probe 40). The mobile clinical viewing device 20 may be or include any mobile, handheld computing device having a display, including, for example, a tablet computer, a smart phone, or the like.

The probe 40 is electrically coupled to the tablet 20 by a cable 12. The cable 12 includes a connector 14 that detachably connects the probe 40 to the tablet 20. The cable 12 facilitates bi-directional communication between the tablet 20 and the probe 40.

In some embodiments, the probe 40 need not be electrically coupled to the tablet 20, but may operate independently of the tablet 20, and the probe 40 may communicate with the tablet 20 via a wireless communication channel.

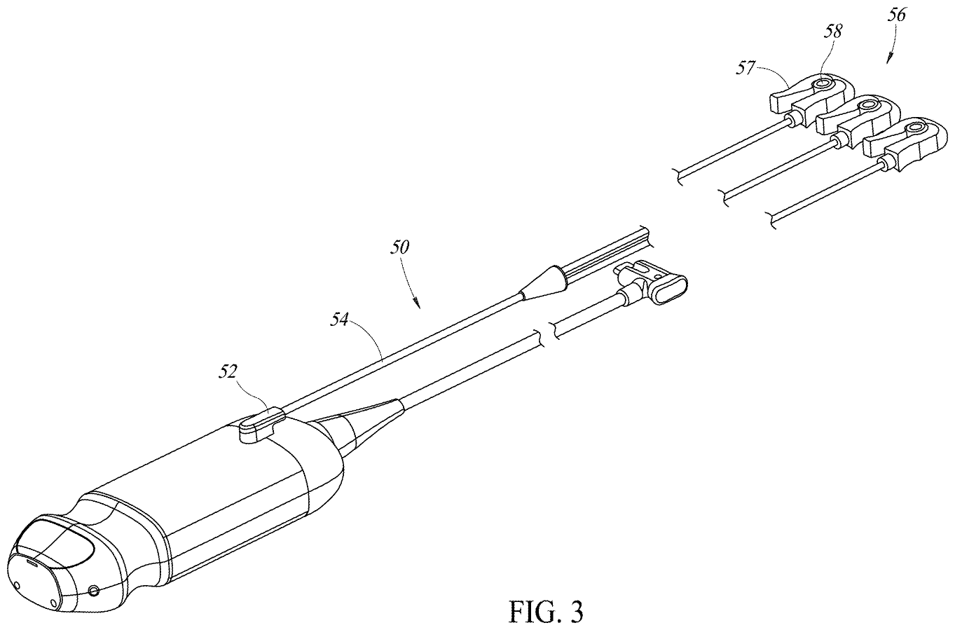

The tablet 20 shown in FIG. 1 includes a display 21. The display 21 may be a display incorporating any type of display technology including, but not limited to, LCD or LED display technology. The display 21 is used to display clinical data acquired by the probe 40. In some embodiments, the probe 40 includes an ultrasound sensor, and the display 21 may be used to display one or more images generated from echo data obtained from the echo signals received in response to transmission of an ultrasound signal. In some embodiments, the display 21 may be used to display color flow image information, for example, as may be provided in a Color Doppler imaging (CDI) mode of ultrasound imaging. Moreover, in some embodiments, the display 21 may be used to display ECG data acquired by one or more ECG sensors (which may be referred to herein as ECG electrodes or ECG leads), which may be or include auxiliary ECG assemblies or leads as will be described in further detail herein with respect to FIGS. 3-12. In some embodiments, the display 21 may be used to display auscultation data, such as audio waveforms representative of auscultation data acquired by one or more auscultation sensors.

In some embodiments, the display 21 may be a touch screen capable of receiving input from a user that touches the screen. In such embodiments, some or all of an external surface of the display 21 may be capable of receiving user input via touch. In some embodiments, the tablet 20 may include a user interface having one or more buttons, knobs, switches, or the like, capable of receiving input from a user of the tablet 20. In some embodiments, the user interface may be at least partially included on the display 21, e.g., with one or more selectable elements visually displayed or displayable on the display 21.

The tablet 20 may further include one or more audio speakers that may be used to output acquired or conditioned auscultation signals, or audible representations of ECG signals or ultrasound echo signals, blood flow during Doppler ultrasound imaging, or other features derived from operation of the system 10.

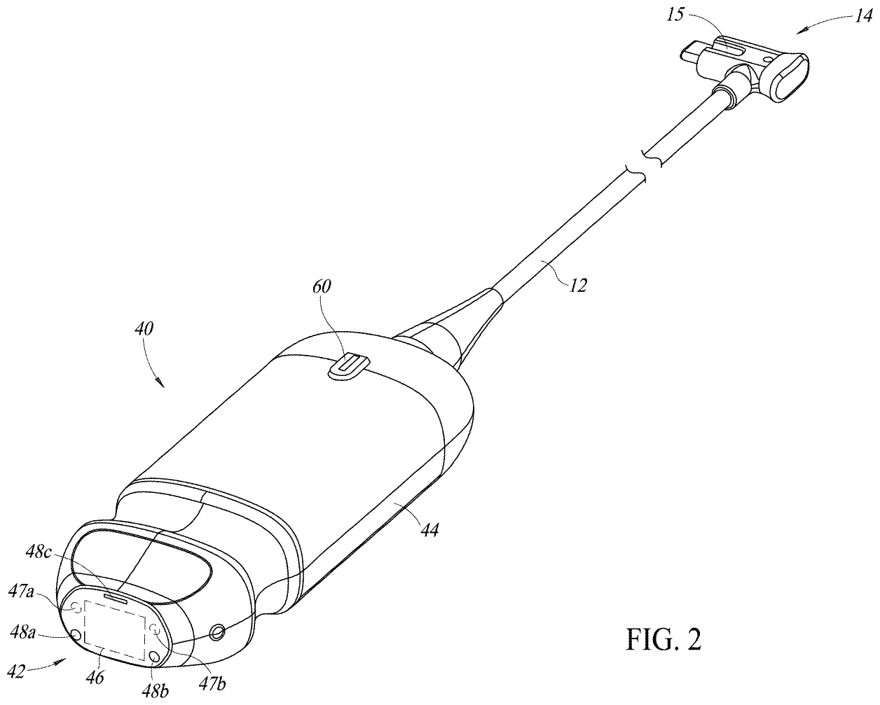

Referring to FIG. 2, the probe 40 includes an outer housing 44 which may surround internal electronic components and/or circuitry of the probe 40, including, for example, one or more ultrasound transducers, electronics such as driving circuitry, processing circuitry, oscillators, beamforming circuitry, filtering circuitry, and the like. The housing 44 may be formed to surround or at least partially surround externally located portions of the probe 40, such as a sensor face 42. The housing 44 may be a sealed housing, such that moisture, liquid or other fluids are prevented from entering the housing 44. The housing 44 may be formed of any suitable materials, and in some embodiments, the housing 44 is formed of a plastic material. The housing 44 may be formed of a single piece (e.g., a single material that is molded surrounding the internal components) or may be formed of two or more pieces (e.g., upper and lower halves) which are bonded or otherwise attached to one another.

The probe 40 includes at least one sensor that, in use, acquires physiological data of a patient. In some embodiments, the probe 40 includes an ultrasound sensor 46. In some embodiments, the probe 40 may include one or more electrocardiogram (ECG) sensors and one or more auscultation sensors. For example, U.S. patent application Ser. No. 15/969,632 (now U.S. Pat. No. 10,507,009) and U.S. patent application Ser. No. 16/593,173, assigned to the assignee of the present disclosure and incorporated by reference herein, describe various embodiments of ultrasound probes having one or more of an ultrasound sensor, an auscultation sensor, and an ECG sensor.

As shown in FIG. 2, the ultrasound sensor 46 is located at or near the sensor face 42. For example, in some embodiments, the ultrasound sensor 46 is located behind the sensor face 42 and may be covered by a material that forms the sensor face 42, such as a room-temperature-vulcanizing (RTV) rubber or any other suitable material. In some embodiments, an ultrasound focusing lens is included at the sensor face 42 and may cover the ultrasound sensor 46. The ultrasound focusing lens may be formed of RTV rubber or any other suitable material.

The ultrasound sensor 46 is configured to transmit an ultrasound signal toward a target structure in a region of interest of a patient, and to receive echo signals returning from the target structure in response to transmission of the ultrasound signal. To that end, the ultrasound sensor 46 may include transducer elements that are capable of transmitting an ultrasound signal and receiving subsequent echo signals. In various embodiments, the transducer elements may be arranged as elements of a phased array. Suitable phased array transducers are known in the art.

The transducer elements of the ultrasound sensor 46 may be arranged as a one-dimensional (1D) array or a two-dimensional (2D) array of transducer elements. The transducer array may include piezoelectric ceramics, such as lead zirconate titanate (PZT), or may be based on microelectromechanical systems (MEMS). For example, in various embodiments, the ultrasound sensor 46 may include piezoelectric micromachined ultrasonic transducers (PMUT), which are microelectromechanical systems (MEMS)-based piezoelectric ultrasonic transducers, or the ultrasound sensor 46 may include capacitive micromachined ultrasound transducers (CMUT) in which the energy transduction is provided due to a change in capacitance.

In some embodiments, the probe 40 includes an integrated electrocardiogram (ECG) sensor 48. The ECG sensor 48 may be any sensor that detects electrical activity, e.g., of a patient's heart, as may be known in the relevant field. For example, the ECG sensor 48 may include any number of electrodes 48a, 48b, 48c, which in operation are placed in contact with a patient's skin and are used to detect electrical changes in the patient that are due to the heart muscle's pattern of depolarizing and repolarizing during each heartbeat.

As shown in FIG. 2, the ECG sensor 48 may include a first electrode 48a that is positioned adjacent to a first side of the ultrasound sensor 46 (e.g., adjacent to the left side of the ultrasound sensor 46, as shown), and a second electrode 48b that is positioned adjacent to a second side of the ultrasound sensor 46 that is opposite to the first side (e.g., adjacent to the right side of the ultrasound sensor 46, as shown). The ECG sensor 48 may further include a third electrode 48c that is positioned adjacent to a third side of the ultrasound sensor 46 (e.g., adjacent to the lower side of the ultrasound sensor 46, as shown). In some embodiments, each of the first, second, and third electrodes 48a, 48b, 48c have different polarities. For example, the first electrode 48a may be a positive (+) electrode, the second electrode 48b may be a negative (−) electrode, and the third electrode 48c may be a ground electrode. The number and positions of the ECG sensor electrodes may vary in different embodiments.

The ECG sensor 48 illustrated in FIG. 2 is integrated into the probe 40, e.g., positioned at or adjacent the sensor face 42. As will be described in further detail with respect to FIGS. 3-12, in various embodiments, auxiliary ECG assemblies are provided that are communicatively coupleable to the probe 40 or the tablet 20. The auxiliary ECG assemblies include one or more auxiliary ECG leads which may be utilized in conjunction with or in place of the integrated ECG sensor 48. In some embodiments, the ECG sensor 48 may be omitted from the probe 40, and the auxiliary ECG assemblies may be placed in contact with the patient's skin and used to detect ECG data of the patient.

In some embodiments, the probe 40 further includes one or more auscultation sensors 47a, 47b at or adjacent to the sensor face 42, as described, for example, in U.S. patent application Ser. No. 16/593,173, which is assigned to the assignee of the present disclosure and incorporated by reference herein. The one or more auscultation sensors 47a, 47b may be any sensors operable to detect internal body sounds of a patient, including, for example, body sounds associated with the circulatory, respiratory, and gastrointestinal systems. For example, the auscultation sensors 47a, 47b may be microphones. In some embodiments, the auscultation sensors 47a, 47b may be electronic or digital stethoscopes, and may include or otherwise be electrically coupled to amplification and signal processing circuitry for amplifying and processing sensed signals, as may be known in the relevant field.

Each of the ultrasound sensor 46, the ECG sensor(s) 48, and the auscultation sensor(s) 47 may be positioned at or adjacent to the sensor face 42 of the probe 40. In some embodiments, two or more of the ultrasound sensor 46, the ECG sensor(s) 48, and the auscultation sensor(s) 47 may be positioned on a same plane, e.g., coplanar with one another at the sensor face 42 of the probe 40. In use, the sensor face 42 may be placed in contact with a patient's skin, and the probe 40 may obtain ultrasound, ECG, and auscultation signals via the ultrasound sensor 46, the ECG sensor 48, and the auscultation sensor 47, respectively. The probe 40 may obtain the ultrasound, ECG, and auscultation signals sequentially or simultaneously in any combination.

Clinical data acquired by the probe 40, such as ultrasound signals, ECG signals, auscultation signals, or any other clinical data or signals, may be transmitted to the tablet 20 via the cable 12 and a connector 14. The cable 12 may extend from the probe 40 (e.g., from a proximal end of the probe 40) and terminates at the connector 14.

The connector 14 may be sized and configured to electrically couple the probe 40 to a corresponding probe connector of the tablet 20. For example, the connector 14 may be keyed or otherwise include features which only allow the connector 14 to fit into the probe connector of the tablet 20 if the connector 14 is properly oriented. For example, as shown in FIG. 2, the connector 14 may include one or more grooves 15 sized to accommodate one or more protrusions of the probe connector.

In some embodiments, the connector 14 may include grooves 15 on upper and lower sides of the connector 14, and each of the grooves 15 may be sized to accommodate a corresponding protrusion of the probe connector. The grooves 15 of the connector 14 may ensure proper orientation of the connector 14 when inserted into the probe connector, as the grooves 15 may allow insertion of the connector 14 into the probe connector in only one orientation. Similarly, the grooves 15 of the connector 14 may prevent the connector 14 from being inserted into any conventional electrical connectors, such as a conventional USB-C connector.

In some embodiments, the signals acquired from the auscultation sensor(s) 47, the ECG sensor(s) 48, and the ultrasound sensor 46 may be simultaneously acquired and synchronized with one another. Moreover, in various embodiments, ECG data or ECG signals acquired from any of the various ECG assemblies and ECG leads described herein (e.g., with respect to FIGS. 3-12) may be acquired simultaneously with and synchronized with signals acquired from the auscultation sensor(s) 47, the ECG sensor(s) 48, and the ultrasound sensor 46. For example, U.S. patent application Ser. No. 15/969,632, assigned to the assignee of the present disclosure and incorporated by reference herein in its entirety, describes various embodiments of devices, systems, and methods in which auscultation data, ECG data, and ultrasound data, which are derived from signals received by an auscultation sensor, an ECG sensor, and an ultrasound sensor, respectively, are synchronized.

The signal acquisition and synchronization techniques described in U.S. patent application Ser. No. 15/969,632 may be modified and implemented in embodiments of the present disclosure for similarly synchronizing the acquired auscultation, ECG, and ultrasound signals, as well as any acquired ambient noise signals, e.g., for noise cancellation. In some embodiments, the acquired auscultation, ECG, and ultrasound signals may be synchronously displayed on the display 21.

The clinical data acquisition system 10 further includes processing circuitry and driving circuitry. In part, the processing circuitry controls the transmission of the ultrasound signal from the ultrasound sensor 46. The driving circuitry is operatively coupled to the ultrasound sensor 46 for driving the transmission of the ultrasound signal, e.g., in response to a control signal received from the processing circuitry. The driving circuitry and processor circuitry may be included in one or both of the probe 40 and the tablet 20. The clinical data acquisition system 10 may further include a power supply that provides power to the driving circuitry for transmission of the ultrasound signal, for example, in a pulsed wave or a continuous wave mode of operation.

As shown in FIG. 2, the probe 40 includes an auxiliary ECG connector 60 which communicatively couples external (e.g., auxiliary) ECG leads to the probe 40. The auxiliary ECG connector 60 is at least partially exposed by the housing 44. For example, the housing 44 may partially surround portions of the auxiliary ECG connector 60, while electrical contacts or other outer portions of the auxiliary ECG connector 60 are uncovered or otherwise exposed by the housing 44. In some embodiments, the auxiliary ECG connector 60 is located near a rear portion of the probe 40 so that, in use, the auxiliary ECG connector 60 is positioned distally with respect to a user's hand while the user is holding the probe 40. In various embodiments, the auxiliary ECG connector 60 may be positioned on any of an upper surface, lower surface, or side surfaces of the probe 40.

The auxiliary ECG connector 60 may at least partially extend into an interior space of the probe 40 and may include one or more electrical contacts that are electrically coupled to circuitry within the probe 40, such as processing circuitry or the like for processing ECG signals received through the auxiliary ECG connector 60. The electrical contacts of the auxiliary ECG connector 60 may be exposed and configured to electrically couple an auxiliary ECG assembly having auxiliary ECG leads or electrodes to the circuitry within the probe 40.

In some embodiments, the auxiliary ECG connector 60 may protrude outwardly from the housing 44 of the probe 40. The auxiliary ECG connector 60 may include one or more protrusions or protruding features which facilitate coupling (e.g., magnetic, mechanical, or electrical coupling) between an auxiliary ECG assembly and the probe 40.

FIGS. 3 through 19 are views illustrating various features relating to auxiliary ECG leads or auxiliary ECG assemblies, and connection of such auxiliary ECG leads or assemblies to a clinical data acquisition probe, such as the probe 40.

ECG voltages measured during routine cardiology examinations are typically on the order of hundreds of microvolts up to several millivolts. Such low voltage ECG signals are generally processed by circuitry such as filter circuitry (e.g., to filter out noise) and amplification circuitry (e.g., to amplify the acquired ECG signal). The ECG sensor 48 including electrodes 48a, 48b, 48c located at or near the sensor face 42 of the probe 40, as shown in FIG. 2, facilitates convenient and useful acquisition of ECG data for various clinical examinations. In some embodiments, the use of auxiliary ECG leads or auxiliary ECG assemblies facilitates acquisition of higher quality and more robust ECG data than would otherwise be attainable through use of only the ECG sensor 48 at the sensor face 42 of the probe 40.

Due to the relative close proximity of the electrodes 48a, 48b, 48c of the ECG sensor 48 at the sensor face 42 of the probe 40, as well as operation of the probe 40 to simultaneously acquire both ECG data and ultrasound data (e.g., ultrasound images), acquisition of high quality ECG data may be challenging in certain circumstances using only the ECG sensor 48. For example, in situations in which an ultrasound gel is used between the sensor face 42 and the skin of the patient during ultrasound imaging, the ultrasound gel (which is typically a water-based gel) may spread across the sensor face 42 of the probe 40 and could potentially “short” the ECG electrodes 48a, 48b, 48c or otherwise reduce the quality of the acquired ECG data or signal.

The use of auxiliary ECG leads, as provided in various embodiments herein, further facilitates ECG data acquisition within a broader or wider anatomical window, as the auxiliary ECG leads may be positioned on dry skin farther apart from one another than the electrodes 48a, 48b, 48c of the ECG sensor 48 at the sensor face 42 of the probe 40.

In various embodiments, the auxiliary ECG assemblies may include any number of ECG electrodes in any desired configuration (e.g., 3 lead, 5 lead, or 12 lead). Transmission of the low voltage ECG signals to the probe 40 may be provided via standard ECG cables or via Bluetooth or similar wireless personal area network (WPAN). This provides a high quality ECG signal while allowing for simultaneous cardiac ultrasound imaging and auscultation signal acquisition.

In various embodiments, the auxiliary ECG assemblies may provide or supplement the ECG heart monitoring capability of the probe 40. In some embodiments, as previously discussed herein, the probe 40 may include ECG electrodes 48a, 48b, 48c, for example, on the sensor face 42 of the probe 40. This allows for simultaneous acquisition of an ECG signal during a diagnostic cardiac imaging session on one integrated device. In some embodiments, however, it may be advantageous for various reasons to include an auxiliary ECG assembly for acquiring ECG signals instead of or in addition to ECG electrodes which may be integrated with the probe 40. For example, in some circumstances, there may be a risk that ECG electrodes on the sensor face 42 of the probe 40 will become electrically short circuited due to the presence of ultrasound gel on the patient which contacts the sensor face 42 of the probe 40. In addition, the anatomical windows for optimal cardiac imaging (e.g., using the probe 40 for cardiac ultrasound imaging) are not necessarily optimal for ECG acquisition. The inclusion of auxiliary ECG assemblies or leads, as provided herein, may therefore reduce or eliminate the possibility of electrical short circuits due to the presence of ultrasound gel and may increase the resolution and fidelity of the ECG signal obtained during such an evaluation.

In various embodiments, the ECG assemblies provided herein may utilize a 3-lead, 5-lead, or any other suitable lead configuration. This may be accomplished with ECG leads and cables (collectively, ECG assemblies) which may be connected to the probe 40 via any suitable connector, which in various embodiments may be, for example, a standard male-female connector, a magnetically coupled connector, an adhesive connector, or a clip-on connector. In some embodiments, the ECG assemblies may be communicatively coupleable to the probe 40 via wireless communication, such as via Bluetooth or other wireless personal area network (WPAN). In some embodiments, in-line electrode pads are provided for communicatively coupling the ECG assemblies to the probe 40.

In some embodiments, magnetic connectors are integrated into the probe 40, which can be coupled to corresponding magnetic connectors of the auxiliary ECG assembly. In some embodiments, ECG assemblies may be of a “snap-on” type and may fit over a distal portion of the probe 40. The snap-on ECG assemblies may electrically insulate the integrated ECG leads of the probe 40 to eliminate shorting. Connectivity to standard ECG electrodes may be made via cables from the snap-on assembly to standard electrode clips.

In some embodiments, the auxiliary ECG assembly or auxiliary ECG leads may be wirelessly coupled to one or both of the probe 40 and the tablet 20. For example, in some embodiments, the auxiliary ECG assembly or auxiliary ECG leads is coupled to one or both of the probe 40 and the tablet 20 through a Bluetooth connection.

Referring now to FIG. 3, an auxiliary ECG assembly 50 is illustrated that is connected to the clinical data acquisition probe 40, in accordance with one or more embodiments. The auxiliary ECG assembly 50 includes a connector 52, a cable 54, and a plurality of ECG leads 56. In use, the ECG leads 56 of the ECG assembly 50 may be positioned on a patient (e.g., on the skin of the patient) and utilized to acquire ECG data (e.g., ECG signals) which are communicated via the cable 54 to the probe 40. The ECG data may be processed, for example, by circuitry within the probe 40 itself, by circuitry within the tablet 20, or by circuitry in a remote electronic device which may communicate (e.g., wirelessly, wired, or the like) with the tablet 20 or the probe 40.

The connector 52 of the auxiliary ECG assembly 50 may be selectively coupled to the auxiliary ECG connector 60 of the probe 40. In some embodiments, the connector 52 may be mechanically and electrically coupled to the auxiliary ECG connector 60. For example, in some embodiments, the connector 52 is sized to snap onto or otherwise snuggly fit over the auxiliary ECG connector 60 such that the connector 52 is not easily or inadvertently removed from the auxiliary ECG connector 60. In some embodiments, one or both of the connector 52 of the auxiliary ECG assembly 50 or the auxiliary ECG connector 60 of the probe 40 includes a magnet for magnetically coupling the connector 52 to the auxiliary ECG assembly 50. The connector 52 of the auxiliary ECG assembly 50 is capable of being selectively attached to and detached from the auxiliary ECG connector 60, for example, by manually attaching or detaching the connector 50. The connector 52 may include one or more electrical contacts that correspond to the electrical contacts of the auxiliary ECG connector 60 on the probe 40.

As shown in FIG. 3, the ECG leads 56 may be of a clip-on type. For example, the ECG leads 56 may include clips 57 that partially surround and are connected to conductive cylinders or sleeves 58. The ECG leads 56 may be configured to clip onto a corresponding conductive post which may be connected to a patch that is applied to a patient's skin at a desired location. In some embodiments, pinching or squeezing the clip 57 may cause a change in a dimension of the conductive sleeve 58. For example, the diameter of the conductive sleeve 58 may be increased in response to a user squeezing the clip 57, which may permit the conductive sleeve 58 to slide over a conductive post (e.g., which may connected to the patient by an adhesive patch or the like). Releasing the clip 57 may cause the conductive sleeve 58 to pinch firmly against the conductive post, e.g., by decreasing the diameter of the conductive sleeve 58.

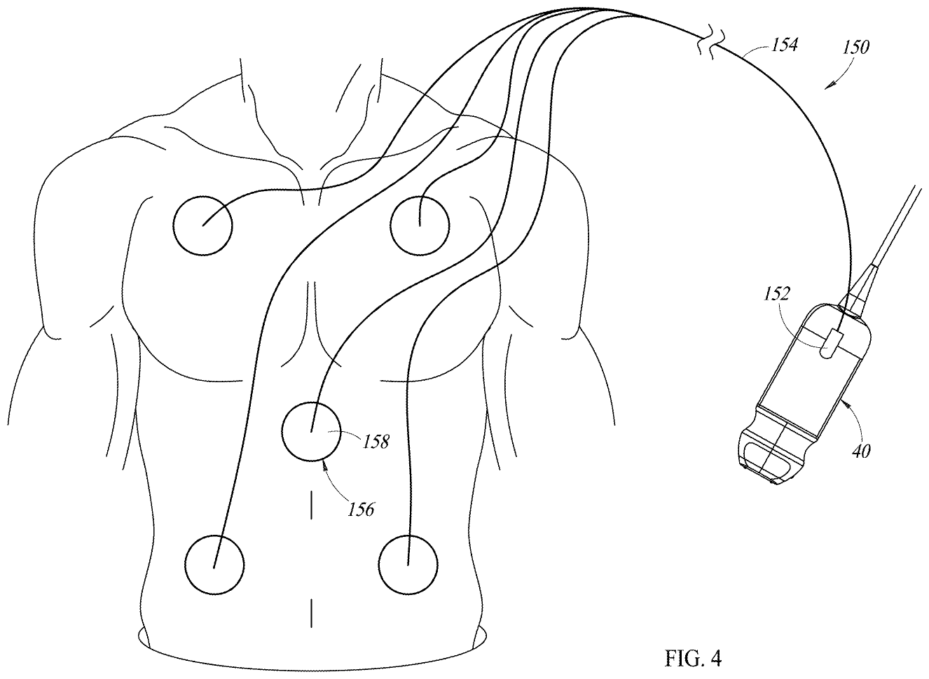

FIG. 4 illustrates an auxiliary ECG assembly 150, in accordance with one or more embodiments. The auxiliary ECG assembly 150 includes a connector 152, cable 154, and ECG leads 156.

The connector 152 and cable 154 of the auxiliary ECG assembly 150 shown in FIG. 4 may be the same or substantially the same as the connector 52 and cable 54 of the auxiliary ECG assembly 50 shown in FIG. 3. One difference, however, is that the auxiliary ECG assembly 150 includes ECG leads 156 which are different from the ECG leads 56 of the auxiliary ECG assembly 50. More particularly, the ECG leads 156 include pads 158 which, in use, are attached to the patient's skin. The pads 158 may be attachable to the patient's skin by any suitable technique. In some embodiments, the pads 158 are adhesive pads that may be adhesively secured at desired locations on the patient. The pads 158 may be electrodes that are electrically connected to respective electrical leads of the cable 154. In some embodiments, the pads 158 may include an electrically conductive material, such as an electrically conductive electrolyte gel, which facilitates electrical conduction from the patient's skin.

In some embodiments, the ECG leads 156 may be disposable leads. For example, the ECG leads 156 may be easily connected to corresponding electrical leads or wires extending from the cable 154. After use during an examination of a patient, the ECG leads 156 may be easily disconnected from the electrical leads or wires and may be disposed. In some embodiments, the entire ECG assembly 150 may be disposable, and the ECG assembly 150 may be disconnected from the probe 40 after use and may be disposed.

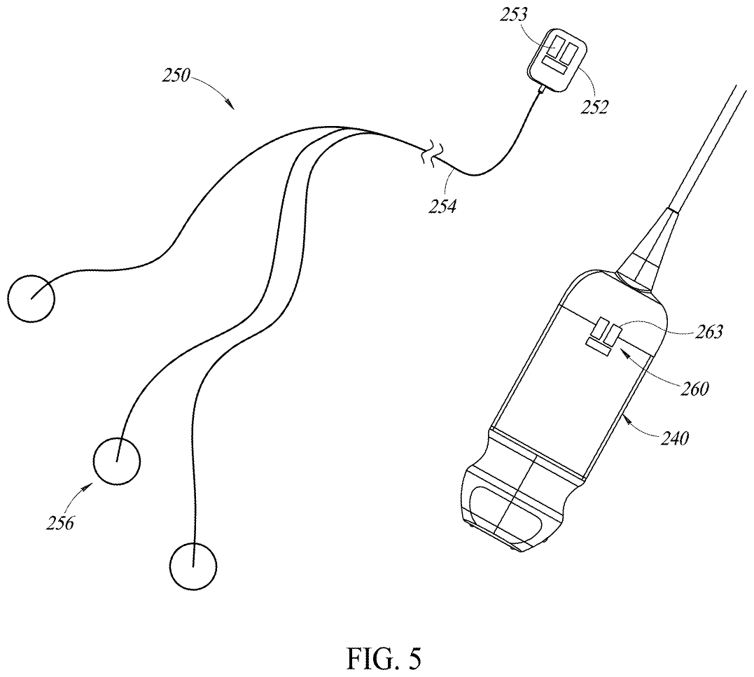

FIG. 5 illustrates an auxiliary ECG assembly 250, in accordance with one or more embodiments. The auxiliary ECG assembly 250 includes a connector 252, cable 254, and ECG leads 256. The cable 254 and ECG leads 256 of the auxiliary ECG assembly 250 shown in FIG. 5 may be the same or substantially the same as the cable 154 and ECG leads 156 of the auxiliary ECG assembly 150 shown in FIG. 4.

The connector 252 of the auxiliary ECG assembly 250 of FIG. 5 is different from the connector 152 of the auxiliary ECG assembly 150 of FIG. 4. In particular, the connector 252 may be an adhesively attachable connector 252. For example, the connector 252 may include one or more electrical contacts 253 and an adhesive (e.g., a medical adhesive) coating over the electrical contacts 253. In some embodiments, the adhesive coating is an electrically conductive adhesive. The adhesive coating is configured to adhere the connector 252 to the auxiliary ECG connector 260 on the probe 240, with the electrical contacts 253 of the connector 252 electrically coupled to corresponding electrical contacts 263 of the auxiliary ECG connector 260.

The probe 240 may be substantially the same as the probe 40 previously described herein, except that the auxiliary ECG connector 260 of the probe 240 may be different as shown in FIG. 5. For example, the electrical contacts 263 of the auxiliary ECG connector 260 may be substantially flush with an outer surface (e.g., the housing) of the probe 240. As such, the auxiliary ECG connector 260 may be free of any plug or other possible entry point for moisture or other contaminants to enter into the housing of the probe 240.

In some embodiments, the auxiliary ECG assembly 250 may be disposable. For example, after use during an examination of a patient, the auxiliary ECG assembly 250 may be easily disconnected from the probe 240 and may be disposed.

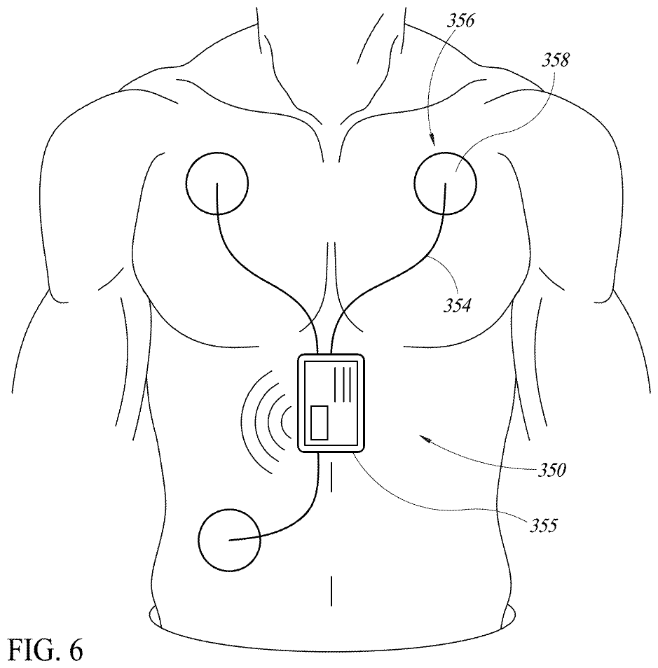

FIG. 6 illustrates an auxiliary ECG assembly 350, in accordance with one or more embodiments. The auxiliary ECG assembly 350 includes a wireless transmitter 355, ECG cables 354, and ECG leads 356.

The ECG leads 356 may be the same or substantially the same as the ECG leads 156 shown and described with respect to FIG. 4. For example, the ECG leads 356 may include pads 358 which are adhesively attachable to the patient's skin. The pads 358 may be electrodes that are electrically connected to respective ECG cables 354.

The ECG cables 354 are electrically coupled to the wireless transmitter 355. The wireless transmitter 355 includes wireless communication circuitry operable to receive ECG data acquired by the ECG leads 356, and to wirelessly transmit the received ECG data to another device. In some embodiments, the probe 40 of the clinical data acquisition system 10 includes wireless communication circuitry operable to receive the ECG data that is wirelessly transmitted from the wireless transmitter 355.

The wireless transmitter 355 may be configured to communicate utilizing any suitable wireless communications technologies or protocols. In some embodiments, the wireless transmitter 355 is a Bluetooth transmitter configured to communicate ECG data using the Bluetooth standard. In some embodiments, the wireless transmitter 355 may be secured to the patient's skin, for example, using an adhesive or the like.

The ECG cables 354 may include electrical output contacts which may be electrically coupled to corresponding input contacts of the wireless transmitter 355. For example, in some embodiments, the ECG cables 354 may include electrical plugs or jacks that may be plugged into corresponding electrical input ports of the wireless transmitter 355. The ECG cables 354 and ECG leads 356 may be disposable after use, while the wireless transmitter 355 may be retained after use for future uses (e.g., by plugging in a new set of ECG cables 354 and ECG leads 356).

In some embodiments, the features and functionality of the wireless transmitter 355 may be incorporated into one or more of the ECG leads 356. For example, each of the ECG leads 356 may include electrodes that are electrically connected to wireless transmitter circuitry that is embedded within, located on, or otherwise mechanically coupled to the pads 358. Each of the ECG leads 356 may wirelessly communicate with the probe 40, and may wirelessly transmit acquired ECG data to the probe 40. In some embodiments, the ECG cables 354 and separate wireless transmitter 355 may be omitted, and the auxiliary ECG assembly 350 may include only the ECG leads 356 having the wireless transmitter 355 integrated therein.

In some embodiments, the ECG leads 356 may include integrated wireless transmission circuitry, and the ECG leads 356 may communicate with a separate wireless transmitter 355. For example, in such embodiments, the wireless transmitter 355 may act as a communications bridge between the ECG leads 356 and the probe 40. The ECG leads 356 may transmit acquired ECG data to the wireless transmitter 355, and the wireless transmitter 355 may in turn collect and transmit the ECG data to the probe 40. In some embodiments, the wireless transmitter 355 may include processing circuitry for processing (e.g., conditioning, amplifying, filtering, synchronizing, etc.) the acquired ECG data received from the ECG leads 356. The wireless transmitter 355 may thus transmit the processed ECG data to the probe 40.

In some embodiments, the auxiliary ECG assembly 350 may include a single ECG lead 356 having an integrated wireless transmitter 355. The ECG lead 356 may include a pad 358 having a plurality of separate embedded electrodes. The pad 358 may have any shape or size. The embedded electrodes of the pad 358 may be spaced apart from one another by any suitable distance for acquisition of ECG data of the patient. The wireless transmitter 355, which is incorporated in the ECG lead 356 (e.g., embedded or located on the pad 358), may be electrically coupled to each of the spaced apart electrodes of the ECG lead 356. As such, the wireless transmitter 355 may be operable to receive ECG data from the electrodes of the ECG lead 356 and to transmit the ECG data to the probe 40.

In various embodiments, the wireless transmitter 355 or integrated wireless transmitter circuitry included within the ECG leads 356 may be formed on a flexible printed circuit board (PCB). Accordingly, the wireless transmitter 355 or integrated wireless transmitter circuitry may be flexible, thereby providing a more comfortable fit when positioned on and adhesively attached to the patient.

In various embodiments, the ECG leads 356 which include integrated wireless transmitter circuitry may include any suitable power source for supplying electrical circuitry for transmitting the acquired ECG data. In some embodiments, the ECG leads 356 including integrated wireless transmitter circuitry may be battery powered, and the batteries may be rechargeable. In some embodiments, the ECG leads 356 may be recharged by placing the ECG leads 356 into a recharging box or case which has electrical contacts configured to supply a recharging current to the ECG leads 356 when positioned within the box or case.

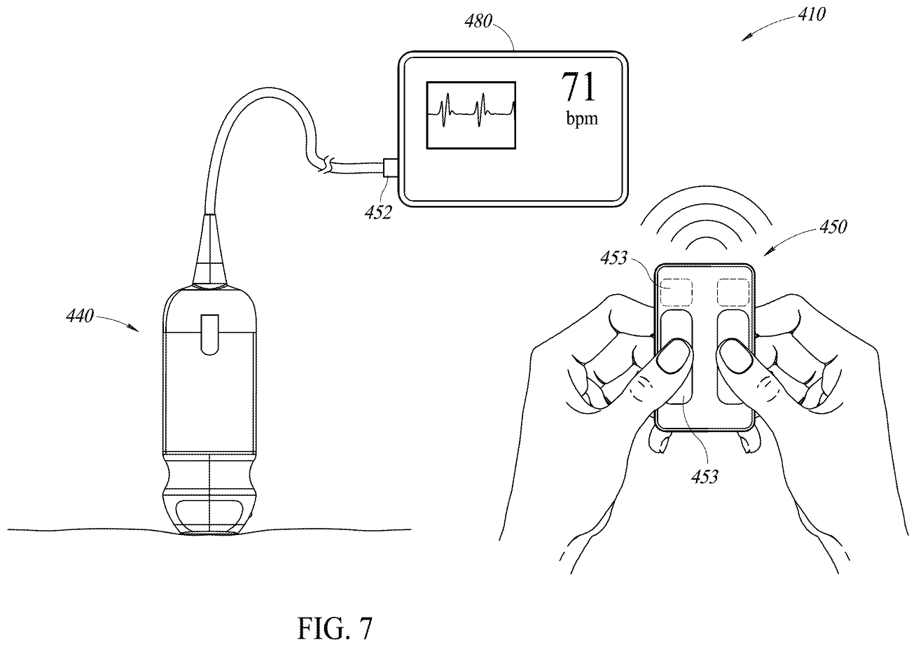

FIG. 7 is a diagram illustrating a clinical data acquisition system 410 including a wireless auxiliary ECG assembly 450, in accordance with one or more embodiments.

The wireless auxiliary ECG assembly 450 may be a handheld unit configured to acquire ECG data from the digits of a user, as shown. For example, the wireless auxiliary ECG assembly 450 may include a plurality of electrical contacts 453 on the front and back sides of the wireless auxiliary ECG assembly 450. In use, the user's thumbs may be placed in contact with electrical contacts 453 located at the front side of the wireless auxiliary ECG assembly 450 and one or more of the user's fingers may be placed in contact with electrical contacts 453 located at the back side of the wireless auxiliary ECG assembly 450. The wireless auxiliary ECG assembly 450 may include circuitry within the assembly that acquires ECG data when the user is holding the assembly as shown.

The clinical data acquisition system 410 further includes a probe 440 and a wireless receiver 480. The probe 440 may be the same or substantially the same as any of the probes previously described herein, such as the probe 40. The probe 440 includes a connector 452 that facilitates electrical coupling with the wireless receiver 480. The connector 452 may be any suitable electrical connector, and in some embodiments the connector 452 may be configured to plug into the wireless receiver 480.

The wireless receiver 480 is configured to receive ECG data from the wireless auxiliary ECG assembly 450. The wireless auxiliary ECG assembly 450 and the wireless receiver 480 may include wireless communication circuitry that facilitates wireless communications utilizing any suitable wireless communications technologies or protocols. In some embodiments, the wireless auxiliary ECG assembly 450 and the wireless receiver 480 are configured to communicate ECG data using the Bluetooth standard.

The wireless receiver 480 may further include a display configured to provide a visual representation of the ECG data received from the wireless auxiliary ECG assembly 450, as shown in FIG. 7. For example, the wireless receiver 480 may display an ECG waveform and may further display a heart rate (e.g., 71 bpm) associated with the received ECG data. The heart rate may be calculated, for example, based on the received ECG data by circuitry located within the wireless receiver 480 or within the wireless auxiliary ECG assembly 450.

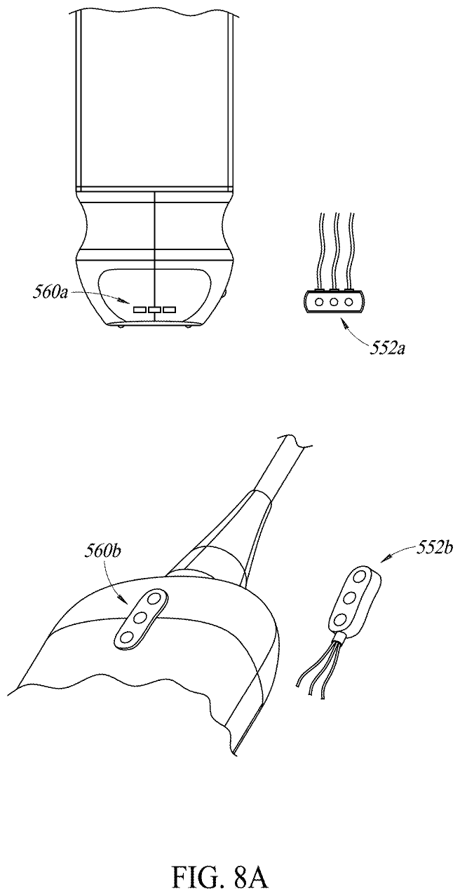

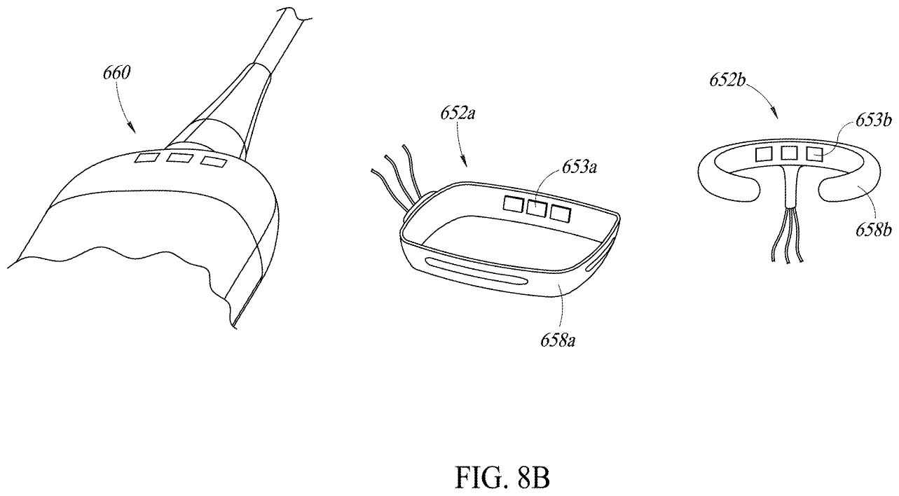

FIG. 8A is a diagram illustrating magnetic connectors for coupling auxiliary ECG assemblies to a clinical data acquisition probe, in accordance with one or more embodiments.

As shown in FIG. 8A, various types of magnetic connectors 552a, 552b may be included as part of any of the ECG assemblies provided herein. The magnetic connectors 552a, 552b may include magnets or magnetic material operable to magnetically secure the magnetic connectors 552a, 552b to a corresponding magnetic ECG connector 560a, 560b of the probe. The magnetic connectors 552a, 552b of the ECG assemblies may have electrical contacts that correspond with electrical contacts of the magnetic ECG connectors 560a, 560b of the probe. The magnetic connectors 552a, 552b may have various different shapes and sizes. The magnetic connectors ECG 560a, 560b of the probe may be located in any suitable position on the probe. For example, the magnetic ECG connector 560a may be located near a distal end of the probe (e.g., near the sensor face), while the magnetic ECG connector 560b may be located near a proximal or rear portion of the probe.

While FIG. 8A illustrates magnetic connectors 552a, 552b, it will be readily appreciated that in various embodiments, the connectors may be selectively secured or securable to the probe by any other suitable technique, including, for example, by an adhesive or the like.

FIG. 8B is a diagram illustrating snap-on type connectors for coupling auxiliary ECG assemblies to a clinical data acquisition probe, in accordance with one or more embodiments.

As shown in FIG. 8B, various types of snap-on connectors 652a, 652b may be included as part of any of the ECG assemblies provided herein. The connectors 652a, 652b may include an outer shell 658a, 658b and electrical contacts 653a, 653b. The electrical contacts 653a, 653b may be formed on inner surfaces of the outer shelves 658a, 658b, as shown.

The outer shells 658a, 658b may be sized to snuggly fit over a portion of the probe including a corresponding ECG connector 660. For example, ECG connector 660 of the probe may be located near a proximal end of the probe, and the outer shells 658a, 658b may include openings configured to slide over or around the proximal end of the probe and to snuggly fit onto the probe with the electrical contacts 653a, 653b being in contact with or electrically coupled to corresponding electrical contacts of the ECG connector 660.

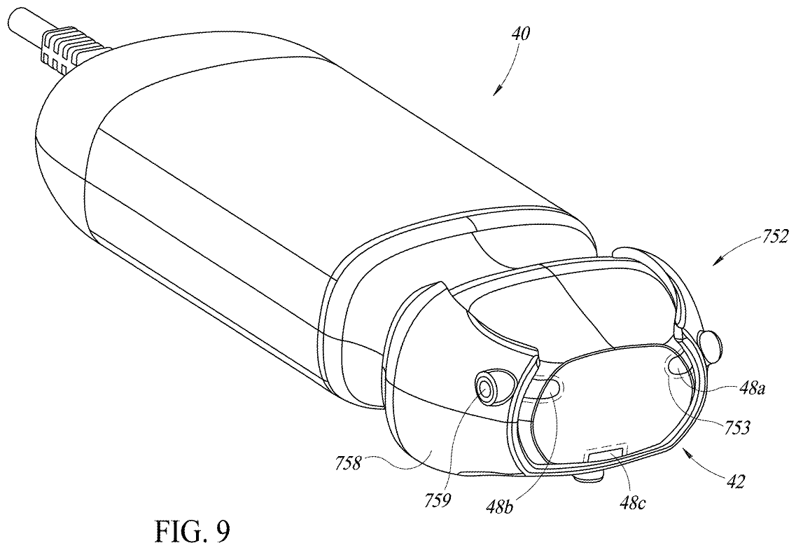

FIG. 9 is a diagram illustrating a snap-on type connector 752 for electrically coupling auxiliary ECG leads to ECG electrodes located at a sensor face of a clinical data acquisition probe 40, in accordance with one or more embodiments.

As shown in FIG. 9, the connector 752 is configured to secure fit onto a distal end of the probe 40, near the sensor face 42. The probe 40 may be the same or substantially the same as the probe 40 previously described herein. As shown, the probe 40 may include ECG electrodes 48a, 48b, 48c located at or near the sensor face 42 of the probe 40. In some embodiments, the ECG electrodes 48a, 48b, 48c may at least partially extend from the sensor face 42 onto lateral or side surfaces connected to the sensor face 42.

The connector 752 includes a shell 758 sized to fit over and provide a snap fit on the distal end of the probe 40, as shown. The connector 752 may include a plurality of electrical contacts 753, each of which may be configured to contact a corresponding one of the ECG electrodes 48a, 48b, 48c when the connector 752 is connected to the probe 40.

In some embodiments, the electrical contacts 753 extend inwardly from the shell 758 and completely cover the corresponding ECG electrodes 48a, 48b, 48c. In some embodiments, an outer or exposed surface of the electrical contacts 753 is covered with an electrically insulating material, which reduces or prevents occurrence of electrical shorts due to the use of ultrasound gel during examination of a patient. When positioned over the probe 40, the connector 752 may cover only the ECG electrodes 48a, 48b, 48c while other sensors at the sensor faced 42 of the probe 40 (e.g., ultrasound sensor and auscultation sensors) may be left uncovered.

Each of the electrical contacts 753 of the connector 752 may be electrically coupled to a respective ECG input port 759. The ECG input ports 759 are configured to receive a corresponding auxiliary ECG wire or lead which may be plugged directly into the ECG input port 759 and electrically coupled to a corresponding ECG electrode 48a, 48b, 48c. Each of the ECG electrodes 48a, 48b, 48c may be electrically coupled to ECG processing circuitry within the probe 40. During operation, the auxiliary ECG wires or leads may be positioned on a patient (e.g., using adhesive pads as described herein, or any other suitable configuration) and ECG data may be transmitted through the ECG input ports 750 to corresponding ECG electrodes 48a, 48b, 48c, and to ECG processing circuitry within the probe 40.

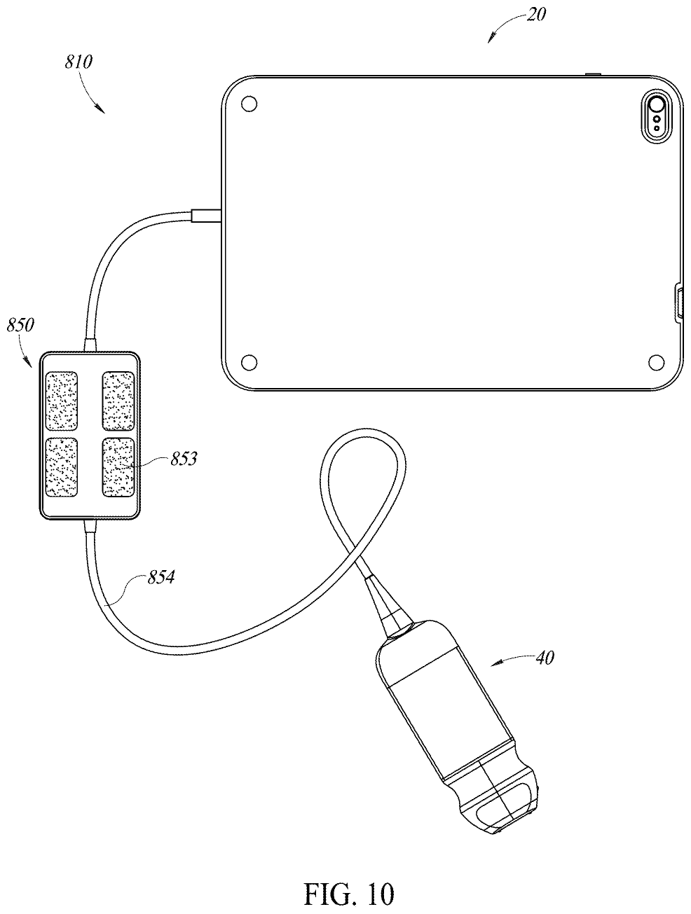

FIG. 10 is a diagram illustrating a clinical data acquisition system 810 including an auxiliary ECG assembly 850 coupled between a mobile clinical viewing device 20 and a clinical data acquisition probe 40, in accordance with one or more embodiments.

The mobile clinical viewing device 20 and the probe 40 may be the same or substantially the same as previously described with respect to any of the various embodiments provided herein.

The auxiliary ECG assembly 850 is electrically coupled to portions of the cable 854 between the mobile clinical viewing device 20 and the probe 40. The auxiliary ECG assembly 850 may include a plurality of ECG contacts 853 operable to receive ECG data and transmit the ECG data to one or both of the mobile clinical viewing device 20 and the probe 40.

In some embodiments, one or more ECG leads or wires are configured to be attached and electrically coupled to the ECG contacts 853 on the auxiliary ECG assembly 850. For example, the ECG contacts 853 may be substantially flat electrical contacts or pads, and auxiliary ECG leads or wires may be adhesively and electrically coupled to the ECG contacts 853. The auxiliary ECG leads or wires may include conductive pads or the like that are positioned at desired locations on a patient to acquire ECG data.

In some embodiments, the ECG contacts 853 of the auxiliary ECG assembly 850 may be extended outwardly from a main body of the auxiliary ECG assembly 850, so the ECG contacts 853 may themselves be brought into contact with the patient. For example, the ECG contacts 853 may include electrical or conductive pads that are connected to lengths of electrical wire, and the pads may be extended outwardly from the main body of the auxiliary ECG assembly 850 and positioned as desired on the patient.

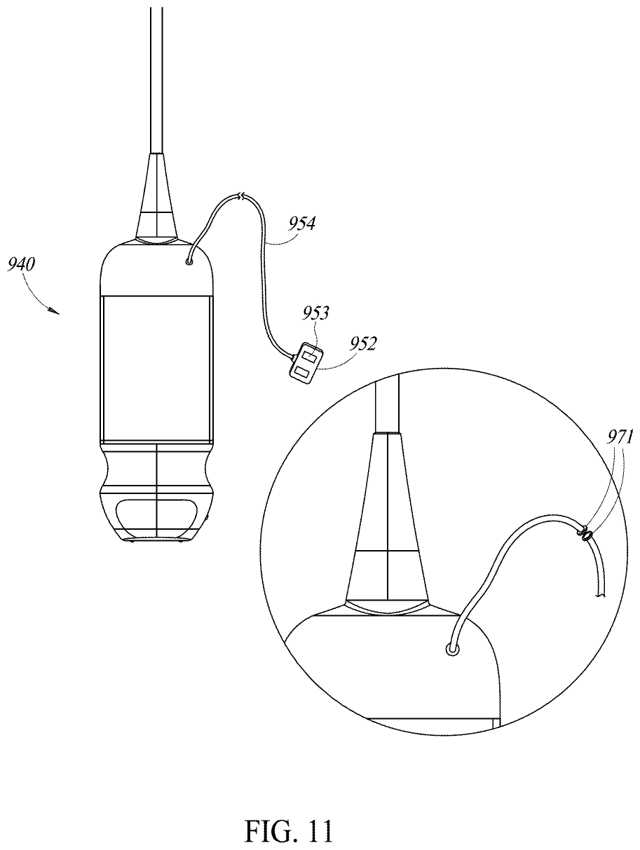

FIG. 11 is a diagram illustrating a clinical data acquisition probe 940 including an auxiliary ECG electrode connector 952, in accordance with one or more embodiments.

The probe 940 may be substantially the same as any of the clinical data acquisition probes previously described herein, except the probe 940 includes an auxiliary ECG electrode connector 952 that is connected to the probe by a cable 954. The ECG electrode connector 952 may include electrical contacts 953 that may be utilized to electrically couple the ECG electrode connector 952 to an auxiliary ECG assembly having electrical leads, pads, or the like that may be attached at desired locations on a patient.

In use, the electrical contacts 953 may receive ECG data acquired by the auxiliary ECG assembly, and may transmit the ECG data to the probe 940 via the cable 954. In some embodiments, the cable 954 may be a continuous electrical cable that extends between the ECG electrode connector 952 and the probe. In other embodiments, the cable 954 may include two or more lengths of electrical cable that may be magnetically coupled together with one or more magnetic connectors 971. The magnetic connectors 971 may physically and electrically couple the separate lengths of electrical cable to one another. The magnetic connectors 971 facilitate easy and convenient detachment of the ECG electrode connector 952 from the probe 940, which may be desirable for examinations using the probe 940 in which ECG data is not needed or in which a longer or shorter electrical cable is appropriate.

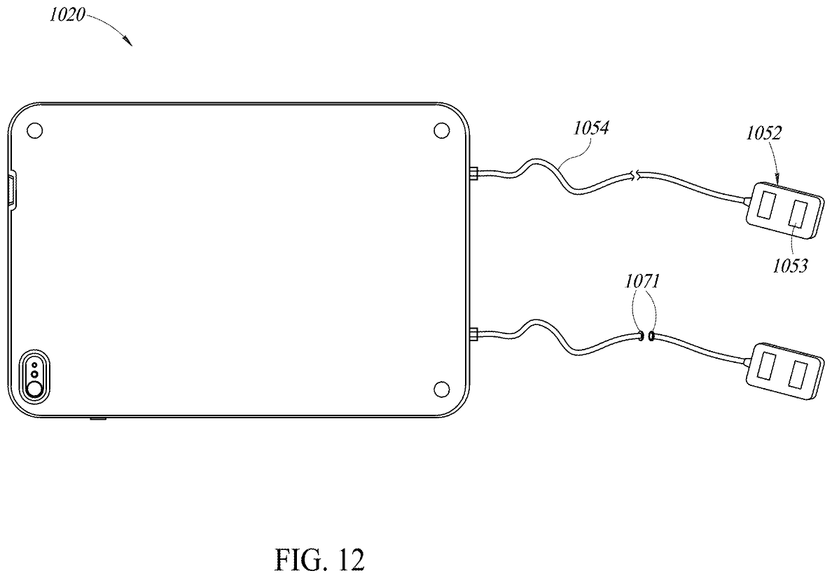

FIG. 12 is a diagram illustrating a mobile clinical viewing device 1020 including an auxiliary ECG electrode connector 1052, in accordance with one or more embodiments.

The may be mobile clinical viewing device 1020 may be substantially the same as the mobile clinical viewing device 20 previously described herein, except the mobile clinical viewing device 1020 includes an auxiliary ECG electrode connector 1052 that is connected to the mobile clinical viewing device 1020 by a cable 1054. The ECG electrode connector 1052 may be the same or substantially the same as the ECG electrode connector 952 described with respect to FIG. 11, and may include electrical contacts 1053 that may be utilized to electrically couple the ECG electrode connector 1052 to an auxiliary ECG assembly having electrical leads, pads, or the like that may be attached at desired locations on a patient.

In some embodiments, the cable 1054 may be a continuous electrical cable that extends between the ECG electrode connector 1052 and the mobile clinical viewing device 1020. In other embodiments, the cable 1054 may include two or more lengths of electrical cable that may be magnetically coupled together with one or more magnetic connectors 1071. The magnetic connectors 1071 may physically and electrically couple the separate lengths of electrical cable to one another.

As may be appreciated by persons having ordinary skill in the art, aspects of the various embodiments described above can be combined to provide further embodiments. Aspects of the embodiments can also be modified, if necessary, to employ concepts of various patents, applications and publications in the relevant art to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A handheld probe, comprising:
a housing;
an ultrasound sensor at least partially surrounded by the housing; and
an auxiliary electrocardiogram (ECG) connector at least partially exposed by the housing, the auxiliary ECG connector configured to electrically couple one or more auxiliary ECG leads to the handheld probe.

2. The handheld probe of claim 1 wherein the auxiliary ECG connector includes a magnet configured to magnetically couple the one or more auxiliary ECG leads to the handheld probe.

3. The handheld probe of claim 1 wherein the auxiliary ECG connector includes a protrusion configured to facilitate a snap fit with a connector that is electrically coupled to the one or more auxiliary ECG leads.

4. The handheld probe of claim 1 wherein the handheld probe is configured to simultaneously acquire ultrasound data via the ultrasound sensor and ECG data via the one or more auxiliary ECG leads.

5. The handheld probe of claim 1, further comprising:
an auscultation sensor,
wherein the handheld probe is configured to simultaneously acquire ultrasound data via the ultrasound sensor, ECG data via the one or more auxiliary ECG leads, and auscultation data via the auscultation sensor.

6. The handheld probe of claim 1, further comprising:
a plurality of ECG electrodes at a sensor face of the handheld probe.

7. The handheld probe of claim 1 wherein the auxiliary ECG connector includes electrical contacts disposed substantially flush with an outer surface of the housing.

8. The handheld probe of claim 1 wherein the auxiliary ECG connector is electrically connected to the handheld probe by a cable.

9. A clinical data acquisition device, comprising:
a handheld probe including at least one sensor configured to acquire physiological data of a patient; and
an auxiliary electrocardiogram (ECG) assembly including a plurality of ECG leads configured to acquire ECG data of the patient, the auxiliary ECG assembly being communicatively coupleable to the handheld probe.

10. The clinical data acquisition device of claim 9 wherein the at least one sensor of the handheld probe includes an ultrasound sensor configured to acquire ultrasound data of the patient.

11. The clinical data acquisition device of claim 9 wherein the handheld probe includes an auxiliary ECG connector, and the auxiliary ECG assembly includes:
a connector; and
a cable electrically connected between the connector and the plurality of ECG leads,
wherein the connector of the auxiliary ECG assembly is configured to electrically couple the plurality of ECG leads to the handheld probe.

12. The clinical data acquisition device of claim 11 wherein each of the ECG leads includes a clip that at least partially surrounds a conductive sleeve.

13. The clinical data acquisition device of claim 11 wherein the connector of the auxiliary ECG assembly is configured be adhesively attached to the auxiliary ECG connector of the handheld probe.

14. The clinical data acquisition device of claim 9 wherein the auxiliary electrocardiogram (ECG) assembly includes a wireless transmitter that is electrically coupled to the auxiliary ECG leads, the wireless transmitter configured to wirelessly transmit the acquired ECG data to the handheld probe.

15. The clinical data acquisition device of claim 14 wherein the wireless transmitter is configured to wirelessly communicate with the handheld probe via Bluetooth.

16. The clinical data acquisition device of claim 14 wherein the ECG leads are located on the wireless transmitter.

17. The clinical data acquisition device of claim 9 wherein the auxiliary ECG assembly is electrically connected between a first cable and a second cable, the first cable being electrically connected to the handheld probe, the second cable operable to electrically couple the ECG assembly and the handheld probe to a mobile computing device.

18. The clinical data acquisition device of claim 9 wherein the handheld probe includes a plurality of ECG electrodes located at a sensor face of the handheld probe, and the auxiliary ECG assembly includes:
a shell sized to fit on the handheld probe;
a plurality of electrical contacts configured to contact the plurality of ECG electrodes when the shell is fit on the handheld probe; and
a plurality of ECG input ports electrically connected to the plurality of electrical contacts, each of the ECG input ports configured to receive a respective one of the ECG leads.

19. A clinical data acquisition system, comprising:
a handheld probe including at least one sensor configured to acquire physiological data of a patient;
an auxiliary electrocardiogram (ECG) assembly including a plurality of ECG leads configured to acquire ECG data of the patient, the auxiliary ECG assembly being communicatively coupleable to the handheld probe; and
a mobile clinical viewing device communicatively coupled to the ultrasound probe, the mobile clinical viewing device including a display configured to display the acquired physiological data of the patient and the acquired ECG data of the patient.

20. The clinical data acquisition system of claim 19 wherein the auxiliary ECG assembly is communicatively coupled to the ultrasound probe by at least one of a male-female connector, a magnetically coupled connector, an adhesive connector, or a clip-on connector.
